(19) United States
(12) Patent Application Publication
(10) Pub. No.: US 2011/0201924 A1
(43) Pub. Date: Aug. 18, 2011

(54) Method and Apparatus for Improving Image Clarity and Sensitivity in Optical Tomography Using Dynamic Feedback to Control Focal Properties and Coherence Gating

(75) Inventors: Guillermo J. Tearney, Cambridge, MA (US); Brett Eugene Bouma, Quincy, MA (US)

(73) Assignee: The General Hospital Corporation, Boston, MA (US)

(21) Appl. No.: 12/845,575

(22) Filed: Jul. 28, 2010

Related U.S. Application Data
(63) Continuation of application No. 10/136,813, filed on Apr. 30, 2002.

(51) Int. Cl. A61B 6/00

(52) U.S. Cl. 600/425

(57) ABSTRACT

Methods for optical imaging, particularly with optical coherence tomography, using a low coherence light beam reflected from a sample surface and compared to a reference light beam, wherein real time dynamic optical feedback is used to detect the surface position of a tissue sample with respect to a reference point and the necessary delay scan range. The delay is provided by a tilting/rotating mirror actuated by a voltage adjustable galvanometer. An imaging probe apparatus for implementing the method is provided. The probe initially scans along one line until it finds the tissue surface, identifiable as a sharp transition from no signal to a stronger signal. The next time the probe scans the next line it adjusts the waveform depending on the previous scan. An algorithm is disclosed for determining the optimal scan range.

### CROSS-REFERENCE TO RELATED APPLICATION

The present application claims priority from copending provisional application No. 60/287,477, filed Apr. 30, 2001, and commonly assigned to the assignee of the present application, and which is incorporated herein in its entirety.

### FIELD OF THE INVENTION

The present invention relates to methods for optical imaging using a low coherence light beam reflected from a sample surface and compared to a reference light beam, wherein real time dynamic optical feedback is used to detect the surface position of a tissue sample with respect to a reference point and the necessary delay scan range. The present also relates to an imaging probe apparatus for implementing the method.

### BACKGROUND

Optical coherence tomography is an imaging technique that measures the interference between a reference beam of light and a detected beam of light that has impinged on a target tissue area and been reflected by scatterers within tissue back to a detector. In OCT imaging of blood vessels an imaging probe is inserted into a blood vessel and a 360 degree circular scan is taken of the vessel wall in series of segments of a predetermined arc to produce a single cross sectional image. The probe tip is rotated axially to create a circular scan of a tissue section and also longitudinally to scan a blood vessel segment length, thus providing two-dimensional mapped information of tissue structure. The axial position of the probe within the lumen remains constant with respect to the axial center of the lumen. However, the surface of the wall may vary in topography or geometry, resulting in the variance of the distance between the probe tip and the surface. Since conventional OCT imaging uses a fixed waveform to create the incident light beam in a schematically rectangular “window” of a certain height, the variation in surface height of the wall may result in the failure to gather tissue data in certain regions of the blood vessel wall. It would desirable to have a feedback mechanism that would cause the modification of the waveform to shift the window based on where the probe is and what it sees.

In traditional OCT systems, the length of the scanning line and its initial position have always been constant and fixed. One way to overcome this problem is to make the window larger. The problem with this is that the signal to noise ratio and accompanying sensitivity decrease because one is collecting information over a larger area in the same amount of time.

It would be desirable to use the identification of the tissue surface to adjust the starting position of the scan to a different spot. The identification of the surface could also be used to adjust the focal location in the sample arm. It would additionally be desirable if the identification of the attenuation of light within the tissue were used to adjust the scan range. The attenuation identification could also be used to determine an optimal depth of focus or confocal parameter.

### SUMMARY OF THE INVENTION

The present invention provides methods for optical imaging using a low coherence light beam reflected from a sample surface and compared to a reference light beam, wherein real time dynamic optical feedback is used to detect the surface position of a tissue sample with respect to a reference point and the necessary delay scan range. The present also relates to an imaging probe apparatus for implementing the method. The probe initially scans along one line until it finds the tissue surface, identifiable as a sharp transition from no signal to a stronger signal. The next time the probe scans the next line it adjusts the waveform depending on the previous scan.

The present invention provides a time delay scanning unit as described herein. The present invention also provides a focus adjusting mechanism for an optical scanning system. The present invention also provides a method of time delay scanning to more accurately determine probe to tissue surface distance variations due to surface topography and probe length/design.

The present invention provides a rocking mirror, as one of several novel mechanisms, to create the delay line. A rocking mirror can be moved much faster and more accurately to retain synchronicity with the computer and the scanning probe. The present invention provides an algorithm to determine position to determine the changes to the galvanometric DC offset angle to conform to tissue distance from the probe tip. In addition, the present invention provides dynamic active feedback to alter the galvanometric AC angle to adjust the coherence gate scan depth to contain only useful image information. Finally, the present invention also is capable of using dynamic active feedback to adjust the focusing properties of the catheter (focal length, spot size, and confocal parameter).

These and other objects, features, and advantages of the present invention are discussed or apparent in the following detailed description of the invention, in conjunction with the accompanying drawings and the appended claims.

### BRIEF DESCRIPTION OF THE DRAWINGS

The various features and advantages of the invention will be apparent from the attached drawings, in which like reference characters designate the same or similar parts throughout the figures, and in which:

FIG. 1 is a graph of a seradyne waveform of a conventional DC baseline offset.

FIG. 2A is a graph of the vessel wall offset contour of one contour scan waveform.

FIG. 2B is the normal (constant offset) scanning wave of ΔL_{R}.

FIG. 2C a graph of the superimposition of the contour ΔL of FIG. 2A onto the seradyne waveform of FIG. 2B.

FIG. 2D is the compensated reference arm scan over a period of two axial scans e₁ and e₂.

FIG. 3A is a graph of the scan depth control.

FIG. 3B is a cross-sectional representation of the lumen and the scan range of FIG. 3A.

FIG. 3C is an image of the cross section of an actual scan.

FIG. 4 is a comparison of the traditional OCT image window and a window using the present invention.

FIG. 5 is a graph of the initial offset and Δz the useful scan range.

FIG. 6 is a graph of the modified galvanometric waveform mapped to conform the reference arm delay to the tissue surface contour.

FIGS. 7A-C show successive delay scan lines of the reference arm.

FIG. 8A shows the Δx versus ΔL.

FIG. 8B shows time versus L_{R}.

FIG. 9 shows a flow diagram of the algorithm according to one embodiment of the present invention.

FIG. 10 shows four possible hits of signal threshold strength and potential tissue surface boundary.

FIG. 11 shows a scan line.

FIG. 12 shows the array of the output/storage of the galvanometric waveform to computer memory.

FIG. 13A shows the old and FIG. 13B new window attainable from block 28 of FIG. 9.

FIG. 14 shows a flow diagram for an alternative embodiment of the present invention providing an autofocus algorithm.

FIG. 15 shows an algorithm for confocal parameter adjustment during confocal microscopy analysis.

FIG. 16 shows a schematic of an apparatus according to one embodiment of the present invention.

FIG. 17 shows a schematic of the delay line.

FIG. 18 is a schematic diagram of an alternative system in which the delay line is created by a mirror 84 is reciprocatingly mounted on a linear translator 85.

FIG. 19 is a schematic diagram showing a further alternative system in which a drum 65 controlled by a computer 25.

FIG. 20 illustrates an alternative using an acousto-optic modulator.

FIG. 21 shows a catheter according to the present invention.

FIG. 22 shows a detail of a catheter according to one embodiment of the present invention.

FIG. 22A shows an inset of FIG. 22 illustrating the movement of the lens with respect to the fiber tip.

FIG. 23 is a detail of a catheter design incorporating a balloon or an expansion chamber to control lens-fiber distance offset.

FIG. 24 shows a schematic view of a system for changing focus.

FIG. 25 shows a schematic view of an alternative embodiment system where the fiber-lens separation is fixed and the separation between the lens and the reflector/prism is changed.

FIG. 26 shows a schematic view of a system where the gap between the fiber and a compound lens composed of multiple elements.

### DETAILED DESCRIPTION OF THE EMBODIMENTS

### Offset and Scan Depth Control

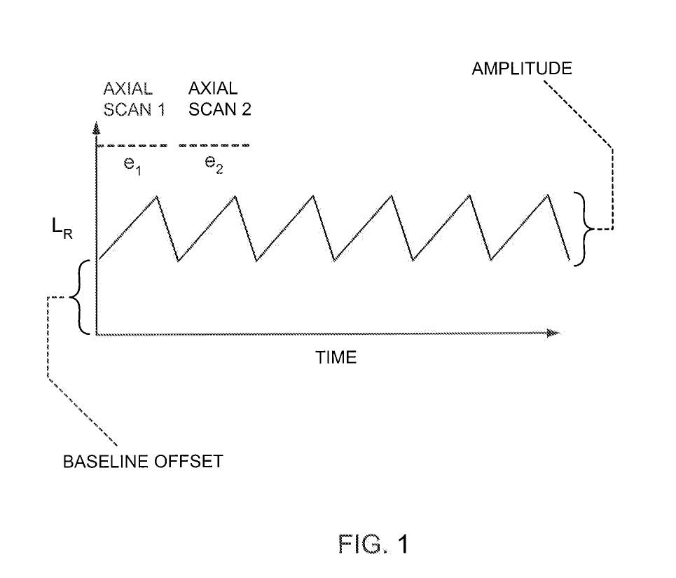

FIG. 1 is a graph of a seradyne waveform of a conventional DC baseline offset, where L_{R} is the reference arm optical delay distance offset and t is time (e.g., 0-20 kHz). One scan image length is shown as “e₁” and a second is shown as “e₂”. The peak-to-peak amplitude is called the AC component.

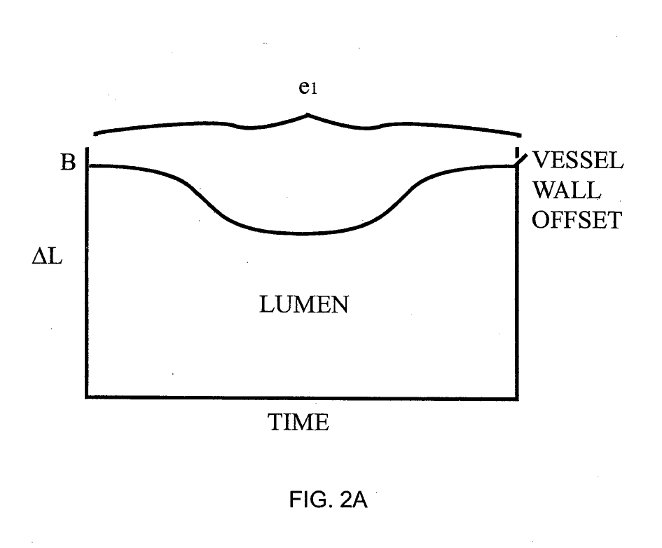

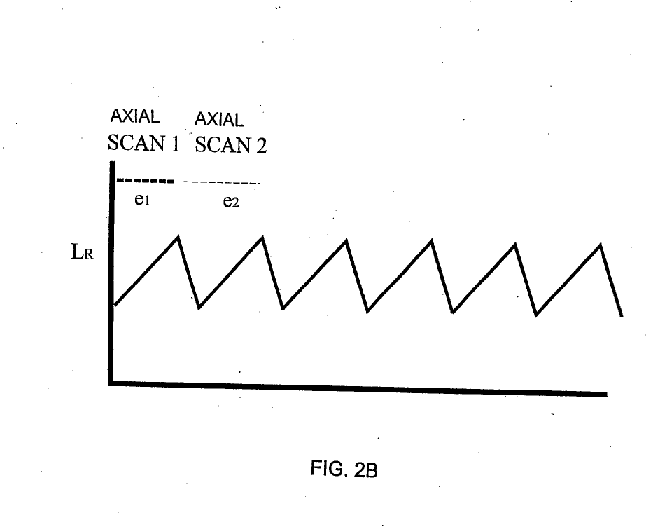

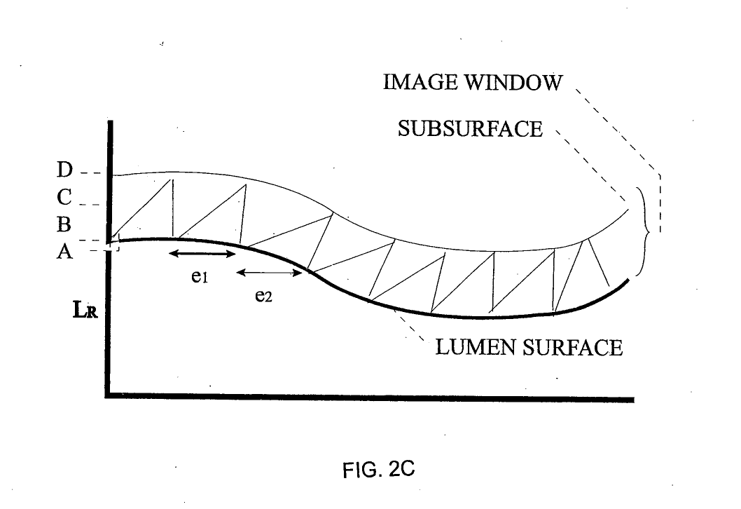

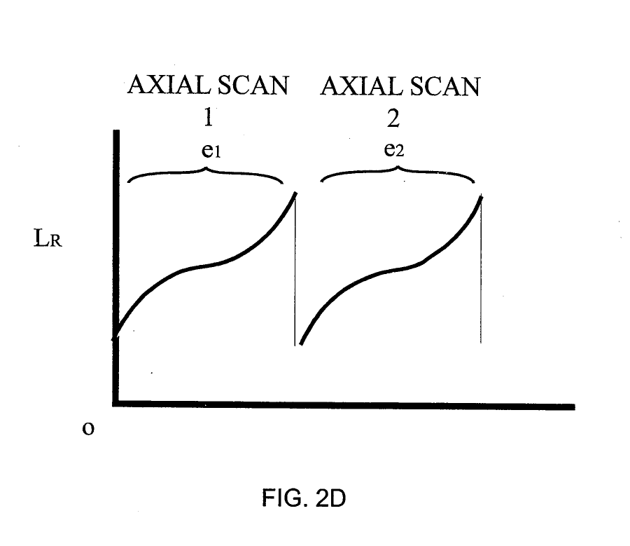

FIG. 2A shows a graph of the vessel wall offset contour of one contour scan waveform where the x-axis is time and the y-axis is ΔL. FIG. 2B shows the normal (constant offset) scanning wave of ΔL_{R} which is a seradyne wave and is shown in where each period is a single scan image (shown as bracketed Axial Scan 1 having a scan image length of e1 and Axial Scan 2 having a scan image length of e2). In a given contour there can be somewhere in the range of 250-500 seradyne scans. FIG. 2A shows the offset correction for a period of a single scan. Optical delay (ΔL) is calculated as

ΔL=ΔL_{S}−ΔL_{R}

where ΔL_{S} is the distance of the sample arm to the tissue surface and ΔL_{R} is the optical path of the reference arm.

FIG. 2C shows the superimposition of the contour ΔL of FIG. 2A onto the seradyne waveform of FIG. 2B. “A” is the start gate; “B” is the tissue or vessel surface; “C” is the inside tissue; “D” is the end gate; and “e” is the waveform period. FIG. 2D shows the compensated reference arm scan over a period of two axial scans e₁ and e₂. A small image window is desirable to reduce signal to noise level. The scan is started at offset “a” (start gate) which is slightly away from the vessel surface so that the vessel surface is at the top of the scan. This is useful in establishing the initial scan offset (starting measurement) for determination of the algorithm (as discussed in detail below). The difference between “b” and “a”, expressed as b−a, is the deadspace between the outside and the vessel surface. b−c is the area inside the vessel surface. The image window of FIG. 2C can be expressed as d−a.

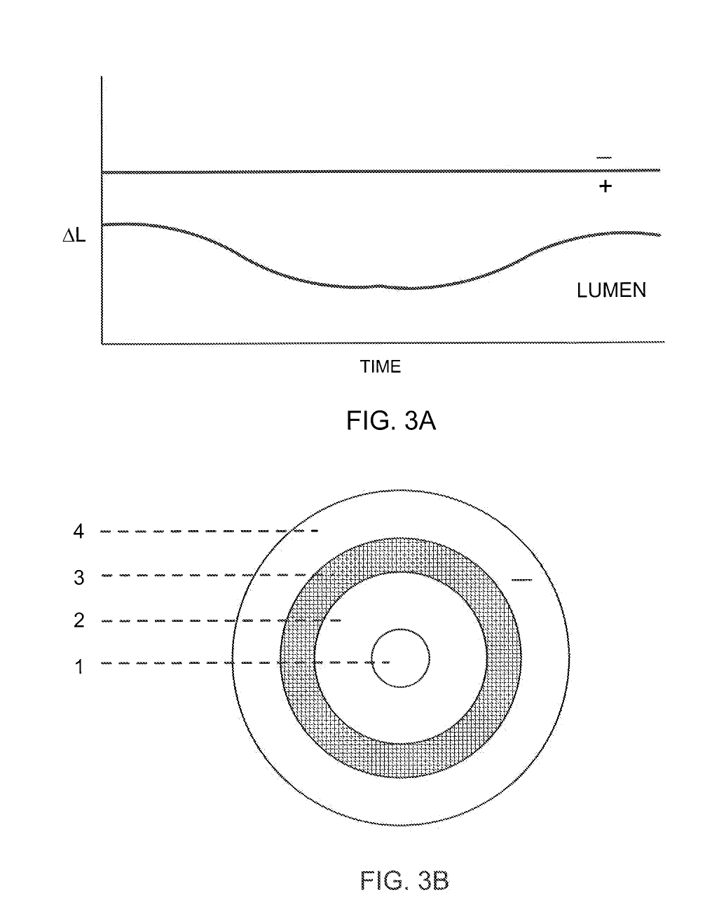

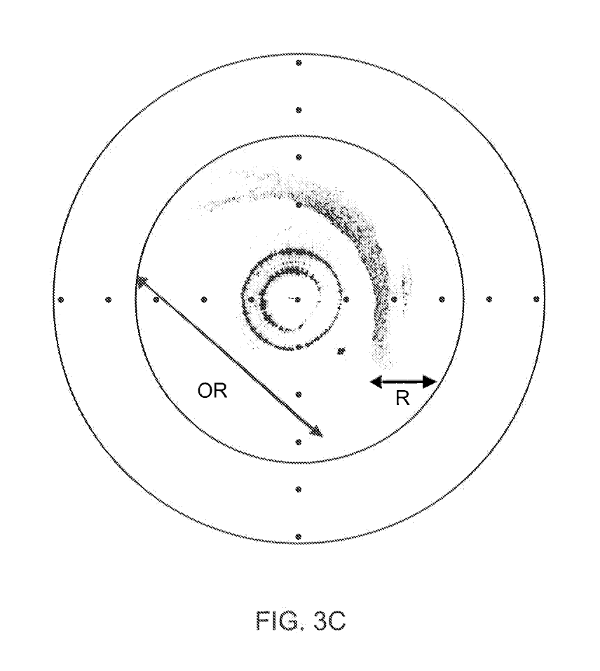

FIG. 3A is a graph of the scan depth control. FIG. 3B is a cross-sectional representation of the lumen and the scan range of FIG. 3A. The innermost circle is the catheter 1, the next circle outward is the vessel lumen 2, the next circle outward is the blood vessel wall 3, and the maximum scan range is indicated at 4. The “+” in a circle area is the useful scan range; the − (minus sign) in a circle is beyond the useful scan range. FIG. 3C is an image of the cross section of an actual scan.

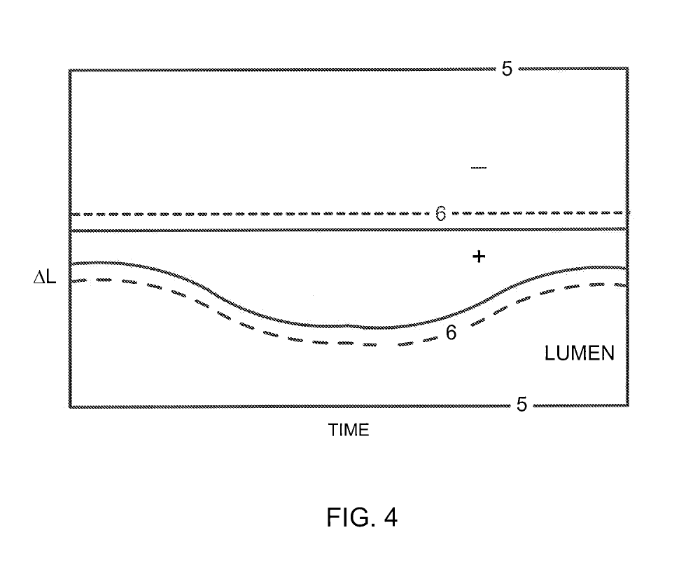

FIG. 4 shows a comparison of the traditional OCT image window, shown as a square labeled 5 (in solid line) and a window obtainable using the algorithm of the present invention where the image window labeled as 6 (in dashed line). The smaller window 6 has much higher signal to noise ratio and therefore provides significantly increased sensitivity, resulting in an improved image quality.

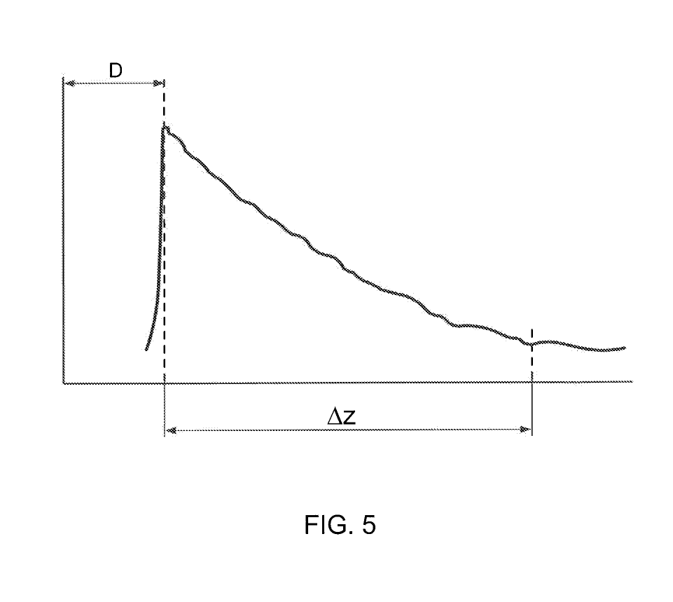

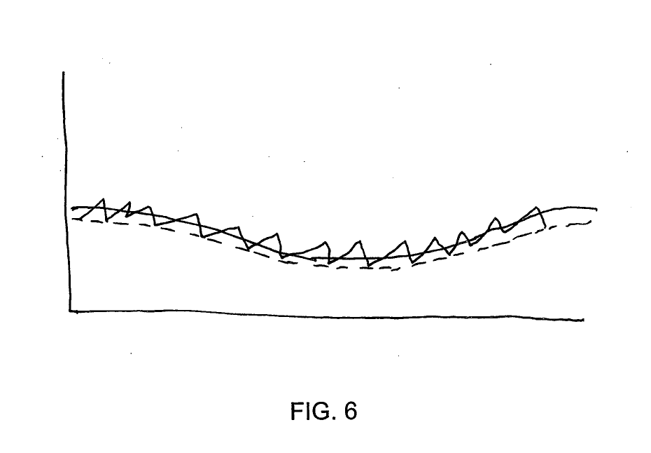

With previous OCT, the scan waveform has a constant AC component and a fixed DC, or slowly varying component. With the present invention the AC component of the waveform as well as the DC component vary with the feedback from the algorithm. See FIG. 5: “D”, the initial offset and Δz the useful scan range is observed to determine how to modify the waveform for the next scan. FIG. 6 is a graph of the modified galvanometric waveform mapped to conform the reference arm delay to the tissue surface contour.

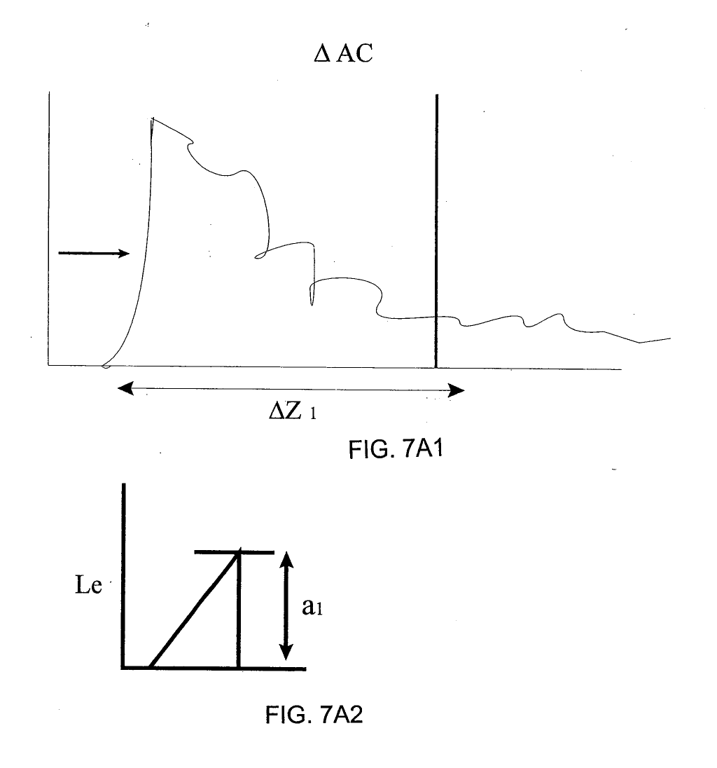

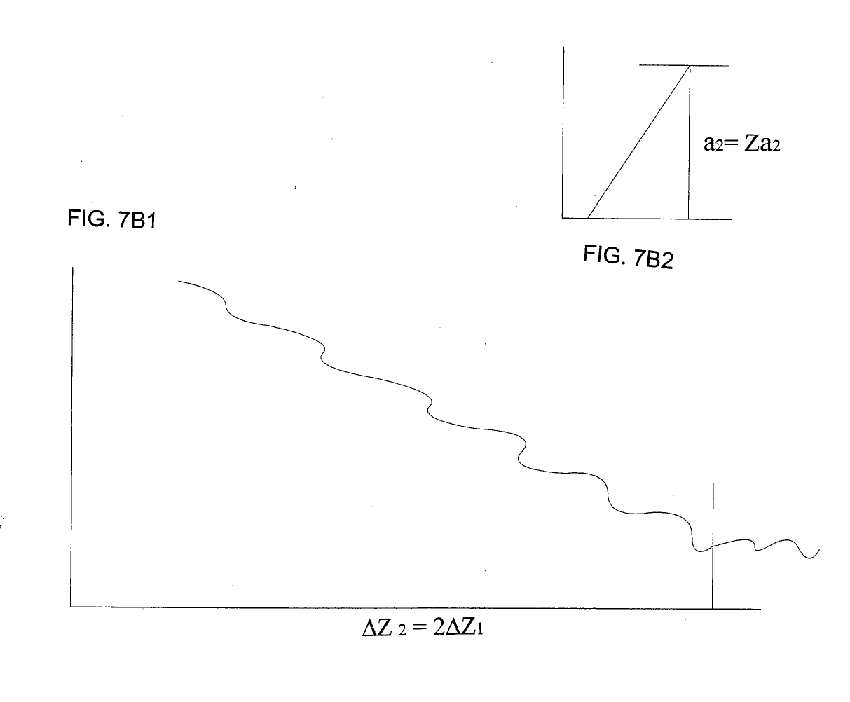

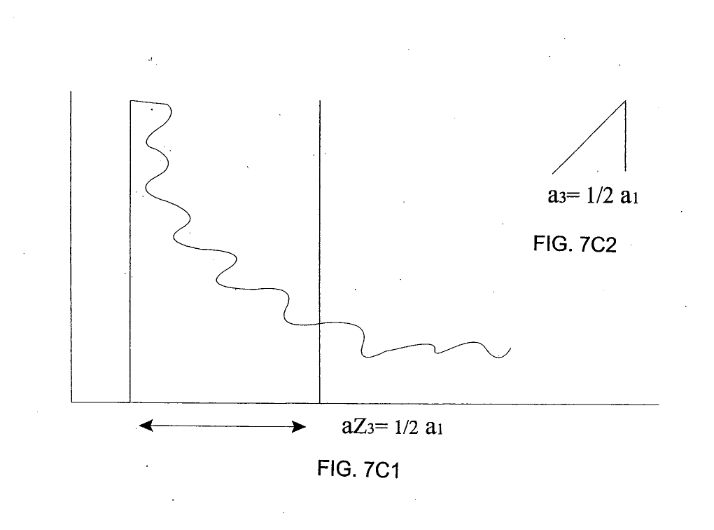

FIGS. 7A-C show successive delay scan lines of the reference arm. FIGS. 7A1 and 7A2 shows amplitude a₁ and Δz₁. FIGS. 7B1 and 7B2 show amplitude a₂=2×a1 and Δz₂=2×Δz₁. FIGS. 7C1 and 7C2 show amplitude a₃=0.5×a₁ and Δz₃=0.5×Δz₁. The longer the range (Δz), the greater the delay in the reference arm.

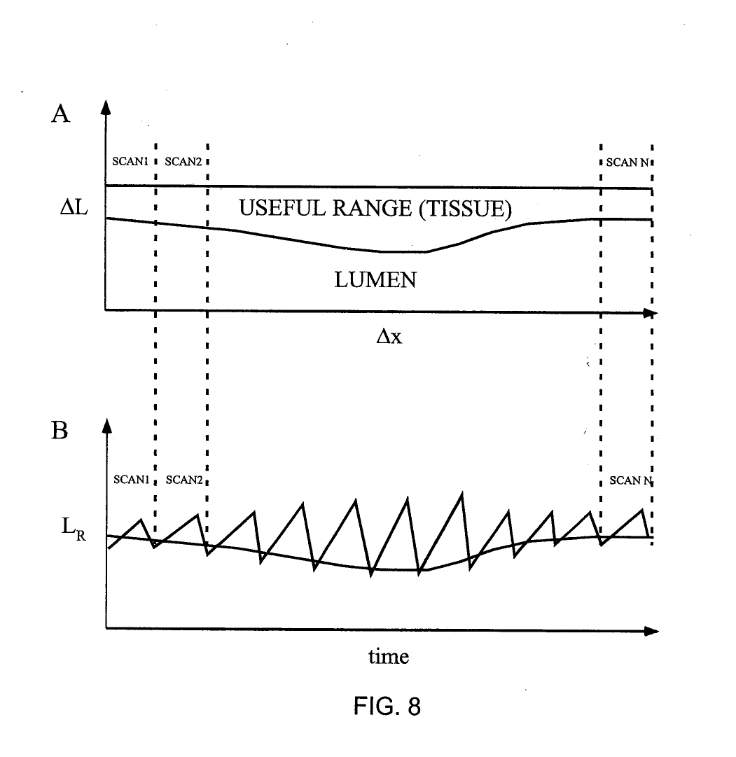

FIG. 8A shows Δx versus ΔL. FIG. 8B shows time versus L_{R}. As the determined scan range increases, the galvanometric reference arm AC component also increases. The DC offset follows the curve representing the tissue surface contour, as in FIG. 8B. Note that Scan 1, Scan 2, etc., of FIG. 8A maps onto Scan 1 and Scan 2 of FIG. 8B. Successive scans 3, 4, . . . N are adjusted for tissue surface offset and optimal scan range in a similar manner. Examination of the data in the present scan line (axial scan) or scan lines determines the offset to the tissue surface and the optimal coherence gate for the following N scan lines. In this manner, real-time dynamic feedback is provided and enables imaging of irregular tissue contours with an optimal sensitivity.

### Method

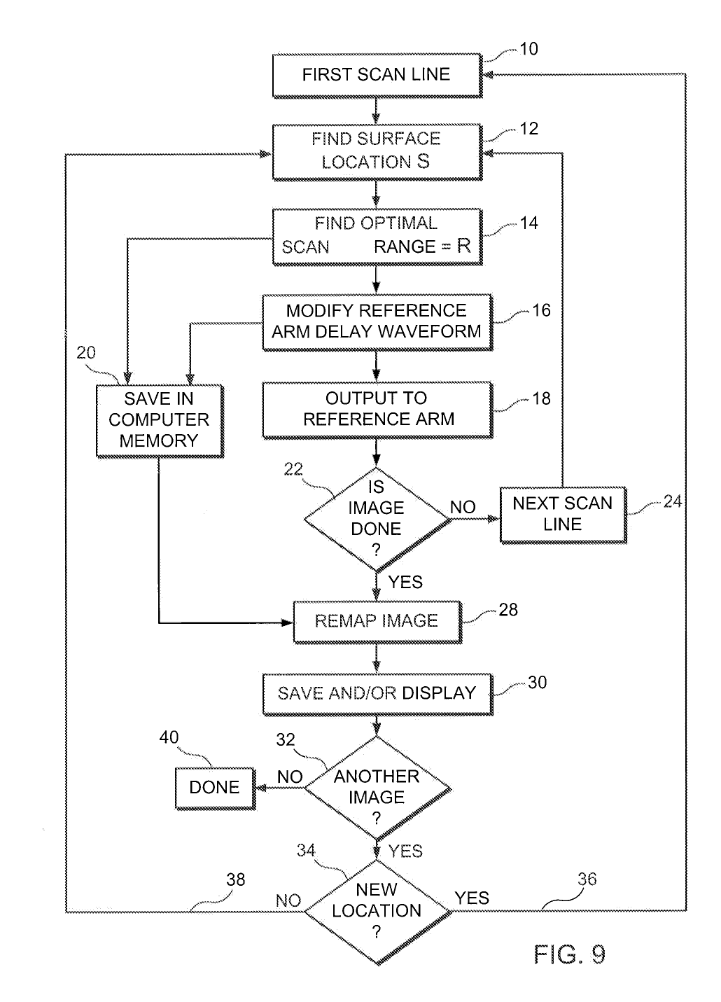

FIG. 9 shows a flow diagram of the algorithm according to one embodiment of the present invention. A first scan line is taken at block 10 sufficient to find the tissue surface “S” at block 12 at a relatively large scan range (block 14) (for example, about 3-10 mm, although other ranges can be used as appropriate). To find the surface one of at least three methods can be used. The first method is to use the adaptive threshold (“T”). The second method uses the first derivative dI(z)/dz=D1. The third method uses the second derivate zero crossing: d²I(z)/dz²=D2.

There are several rules A, B, and C involved. For the first method rule “A” is: if I(z₁)>T, then S=z₁. For the second method, rule “B” is: if dI(z₂)/dz>T, then =z₂S. For the third method, rule “C” is: if d²I(z₃)/dz²=0, then =z₃S. Note, I(z) may need to be filtered to remove noise before doing the derivatives and reduce the introduction of preprocessing spikes. Such filtration may be achieved using any of a number of filters known to those skilled in the art, including, but not limited to, linear blur, Gaussian, windows, low pass filters, convolution, morphology, and the like. If the surface is not found, repeat block 10, but change the range offset based on the results at block 12. For example, if there is no signal, the offset and range may be altered in a random manner. If there is a signal but it is weak and did not exceed an adaptive threshold, the offset is adjusted (i.e., move the S and gate toward the signal and try again). That offset is made based on the intensity of reflect light detected by the detector.

There could be a potential problem at block 12 if the sheath plus internal reflections is catheter based, or signal based, where the highest signal is inside the tissue. In such a case there may be more than one location “z” which has the derivatives >T.

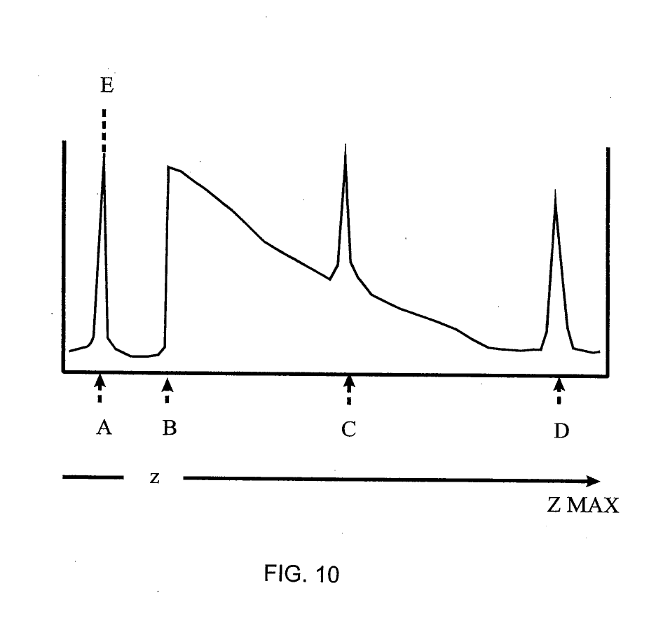

In such cases the rules A, B, and C above are parsed to determine which corresponds to tissue surfaces. FIG. 10 shows four possible hits. There is only one that corresponds to the tissue surface. ε is a small increment. Peak “A” shows an isolated hit where there is no appreciable signal on either side of the peak; therefore, for z_{A}−ε<z_{A}<z_{A}+ε, there is I(z_{A}±ε)<<I(z). Peak “B” shows a peak where there is no signal before (i.e., to the left) but there is signal after (i.e., to the right); therefore, for z_{B}−ε<z_{B} there is I(z_{B}−ε)<<I(z_{B}) and I(z_{B})≈I(z_{B}+ε). Stated differently, FIG. 10 shows four cases where the signal (image data) threshold is exceeded. Peak “A” has no signal before or after it (i.e., within the next pixel, increment or ε) it (sometimes referred to as above (z₀) or below (zₘₐₓ)); therefore, it is discounted. Peak “D” is discounted for the same reason/rule: it has no signal before or after it. For peak “C” there is signal before it and after it, therefore it cannot be at the surface. For peak “B” there is signal after it, but not before it. Therefore, peak “B” indicates the start of the tissue surface boundary.

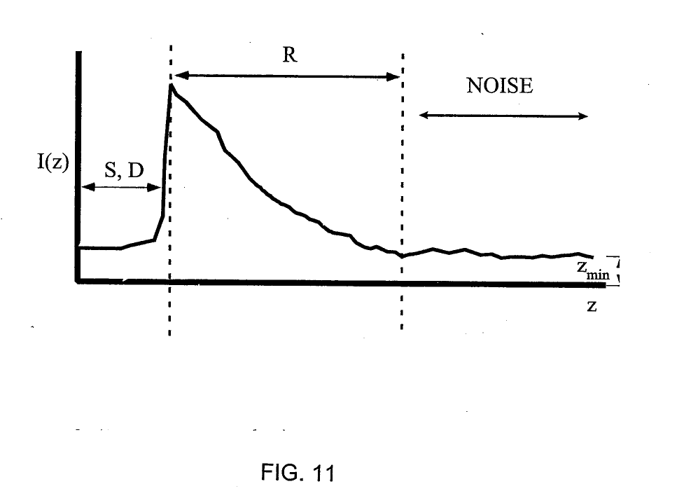

Referring back to block 14 there is now a fixed range, typically larger than desired for the first line. FIG. 11 shows a scan line. The optimal scan range R is what is to be determined. First, the curve is smoothed (see methods mentioned above). Then, second, go out to a large z where there clearly is no signal; i.e., find where I(zₘₐₓ)=noise. This can be verified by finding where the standard deviation of (I(z±ε)) is low. Third, decrease z (i.e., move z towards S) until I(z) starts to increase again; i.e., I(z′)>I(zₘₐₓ) and where R=z′−S.

Another method of achieving a similar result is to first smooth and take the derivative of the curve and find out where d(I(z′))/dz=0 and therefore R=z′−S.

Other statistical methods are possible. A basic operating parameter is that one wants minimal signal outside of and as much signal as possible inside of the scan range R. This can be achieved by zeroth order, first derivative, second derivative, probability distribution functions statistics (e.g., standard deviation), fitting to exponential and other standard data analysis procedures known in the art.

Spikes in noise, but which are artifacts which could be counted in a signal solution can be a potential problem. One can use filters (median, ordered, adaptive, closing, dilitation or other filter known in the art) to eliminate spikes caused by out of range artifacts.

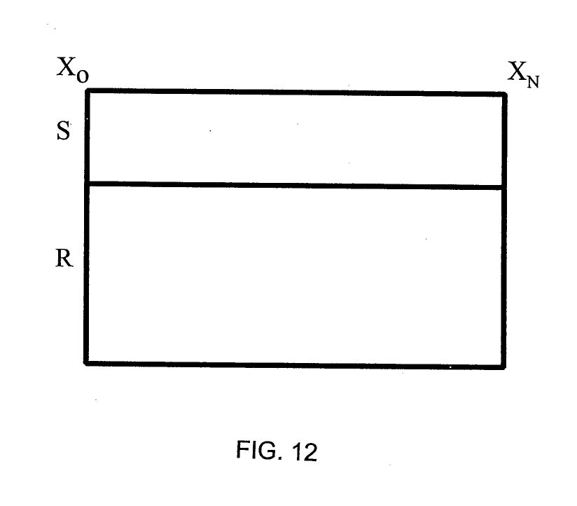

Referring back to FIG. 9, the reference arm delay waveform is modified at block 16. There is a known 1:1 relationship between data acquired by the computer and reference arm position. S and R can be used to modify the waveform controlling the optical delay line. S and R now need to be inserted into an equation which controls the galvanometric waveform. Thus G(t)=f(S,R,t), where G(t) is the galvanometric waveform and f is a function. This G(t) is sent digitally or analog to the galvanometric waveform. FIG. 12 shows the array of the output/storage of the galvanometric waveform to computer memory block 20 and which goes to remapping at block 28, where “N” is the number of axial scans per image. This S,R array indicates how to remap the data into real space again for block 28 (of FIG. 9).

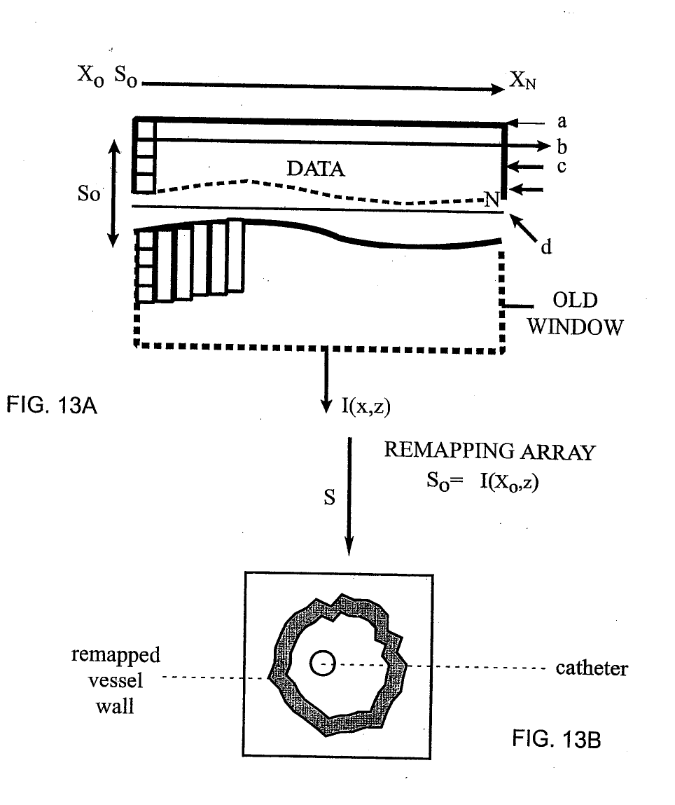

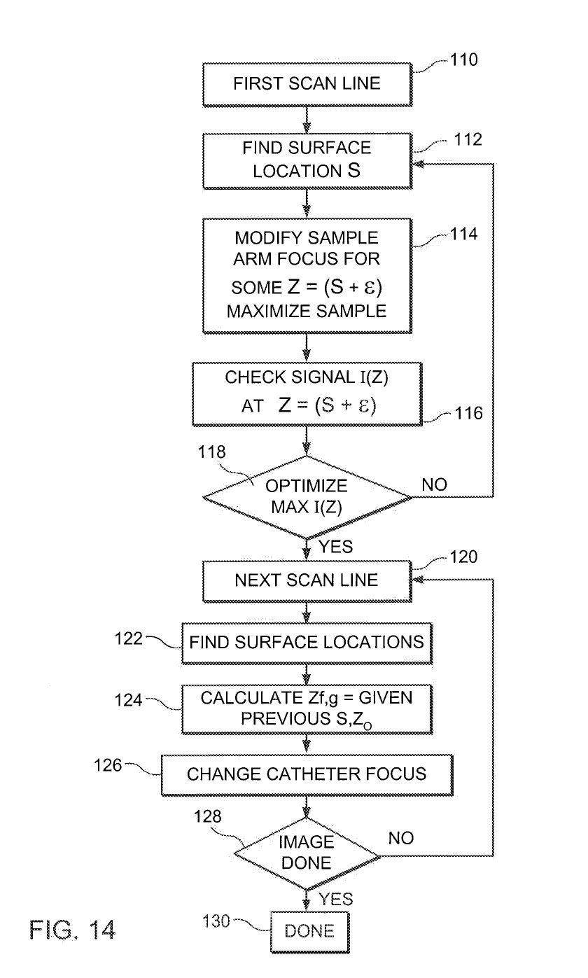

FIG. 13A shows the old and FIG. 14B shows the new window attainable from block 28 (refer back to FIG. 9 and accompanying description of reference letters). I(x,z) are inserted into a remapping function with the inputs being an array of S, R to create the remapped image of block 28. For every line, x, there are different elements, S and R, in the array (i.e., S₀ corresponds to I(x₀,z) and z is continuous. This relates to the distance between the probe and the chosen range.

Remapping (block 28 of FIG. 9) is preferably done after each scan. For storage, the image is remapped after acquisition. For display, remapping is done interactively. Add each S that is known for each of the scan lines (the vertical bars) to the data and the contour is remapped. S is added to the offset of the image. In other words, shifting the data for any given exposition by S. Each vertical bar gets (axial scan) remapped (shifted) based on their respective S value. For example, is the z values in x₁ are offset by S₁.

There are multiple different equations possible for remapping, examples of which are shown below:

I(xₙ,z)=I_{acq}(xₙ,z−Sₙ) (1)

I(xₙ,z)=I_{acq}(xₙ,z−Sₙ₋₁) (2)

I(xₙ,z)=I_{acq}(xₙ,z−Sₙ₊₁) (3)

where n identifies a specific axial scan and where n is close to where mapping is occurring.

One is thus using array R,S to redisplay/remap the image. This is the most efficient way of storing the remapped image. S can be stored +I_{acq}(z) and reconstructed offline. Or, S+I_{acq}(z) can be reconstructed dynamically or interactively.

The output is sent to the reference arm at block 18 and also saved in the computer at block 20. If the image is not done at block 22, the next scan line is taken at block 24 by cycling back repeatedly to block 12 until the image is acquired. If the image is done, then the image is remapped at block 28 using the surface S information and the modified reference arm delay waveform stored and recalled from the computer memory from block 20. The image is then saved or displayed at block 30. If no other image at block 32 is to be taken, the process is done at block 40.

Optionally, if another image is to be taken at block 32, then the algorithm queries at block 34 whether a new location is taken. If yes, then at line 36 the first scan line is taken back at block 10. If no image is scanned at line 38, then the next surface location S is found at block 12.

### Autofocus

In an alternative embodiment the present invention can be used in an autofocus mode. FIG. 14 shows a flow diagram for an autofocus algorithm.

If Sn and Rn are known, then an optimal focal length is also known and the optimal spot size and confocal parameters can be calculated. If some function “g” is applied to the catheter which causes a change in focus by z_{f}, and which occurs at pixel “n” where one knows Sₙ, then all one needs to know is, if one is at Sₖ then one can calculate how g changes as (Sₖ−Sₙ). Therefore, for a given n, one knows what one has to do to the catheter to obtain a focus of z_{f}(n). Sₙ is also known. So, Sₙ₊₁ creates g(n+1) for all n. In other words, S allows one to adjust the focus so that it is optimally present within or at the surface of the tissue. R allows one to adjust the confocal parameter so that the spot size is minimized over the optimal scan range. These alterations of the catheter are performed in real-time, using dynamic feedback obtained from the image. These enhancements enable optimal imaging of the tissue under investigation.

A key feature of the present invention is that one can calculate where to move the focus if one position is known. One does not have to iteratively modify the focus until it is optimized each time, only once, and, once S is calculated, modify focus thereafter using the previous or present S of the scan. The present invention allows imaging of tissue with an irregular surface and keeping substantially the entire image in view. Moreover, the scan range is decreased so as to only include useful image information, therefore decreasing the bandwidth of the signal and increasing the image sensitivity of even possibly up to some 3-5 times. The sensitivity increase may be implemented by decreasing the bandwidth of the filter used reject noise while performing heterodyne or lock-in detection. This filter bandwidth may be adjusted dynamically by using diode switched capacitor arrays. Increasing sensitivity is equivalent to increasing speed while keeping accuracy. This is important in cardiovascular system imaging. Further, increasing speed decreases motion artifacts from heartbeat and blood pressure with concomitant lumen expansion and accompanying modulation of the arm-sample distance. Autofocus enables one to place the optimal focus on the tissue for every scan position in a rapid manner, thus leading to sharper images. The present invention also has the advantage of compensating for probe length variation.

The present invention provides a time delay scanning unit as described herein. The present invention also provides a focus adjusting mechanism for an optical scanning system. The present invention also provides a method of time delay scanning to more accurately determine probe to tissue surface distance variations due to surface topography and probe length/design.

### Confocal Parameter

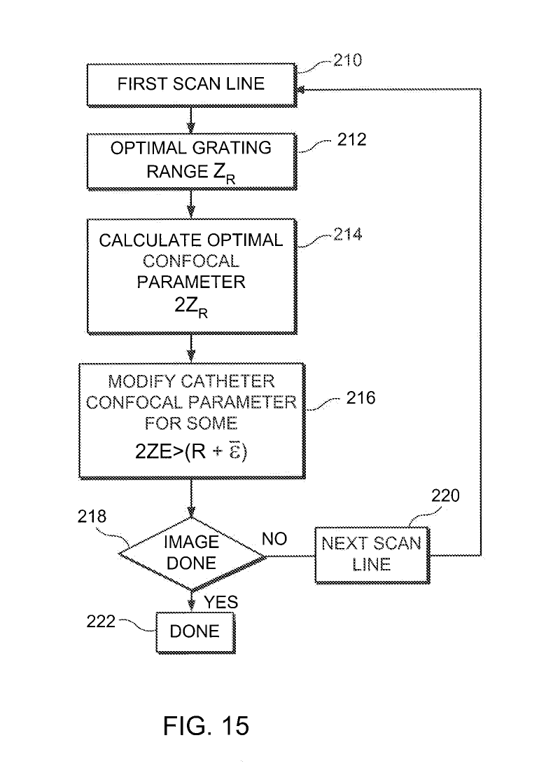

FIG. 15 shows an algorithm for confocal parameter adjustment during confocal microscopy analysis. The confocal parameter is optimized to R, the optimal scan gate range. After the first scan line is taken at block 210, the optimal grating range R (as previously described hereinabove) is determined, block 212. The optimal confocal parameter 2z_{R} is calculated at block 214. Then the catheter confocal parameter is modified at block 216 for some 2zₑ>(R+ε). If the image is not done at block 218, go to the next scan line 220. If the scan is done, end at block 222. 2z_{R}=(2πω₀²)/λ, where ω₀ is the beam radius; λ is wavelength, and 2z_{R} is the confocal parameter.

### Apparatus

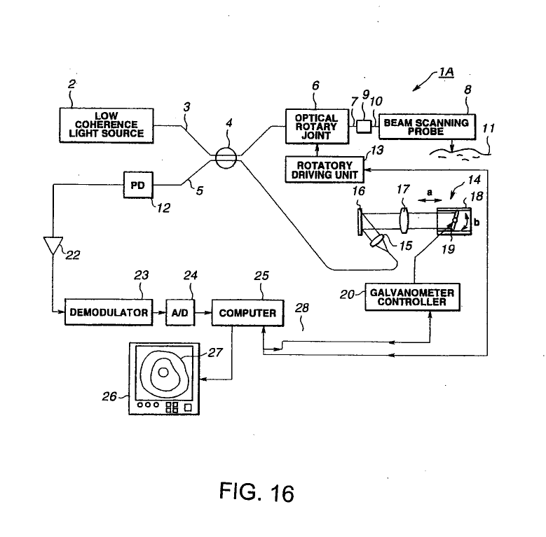

FIG. 16 shows a schematic of an apparatus according to one embodiment of the present invention. The basic description of this and the subsequent drawings is found in Ozawa et al., U.S. Pat. No. 6,069,698, which is incorporated herein. The basic description of the relevant parts of FIG. 16 corresponds to FIG. 1 of Ozawa et al.

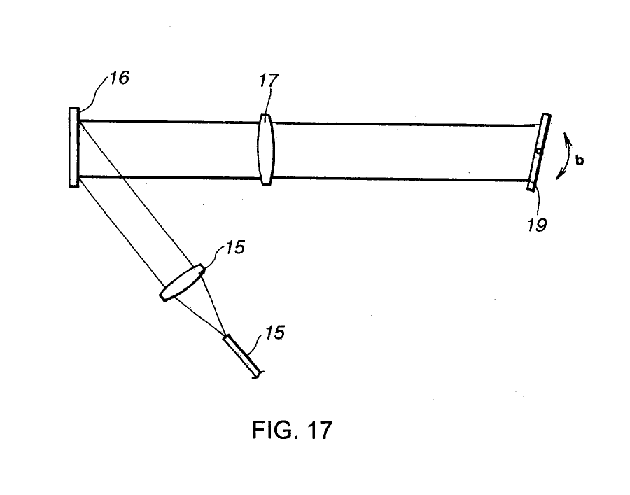

FIG. 17 shows a schematic of the delay line. The galvanometer is a motor that attaches to the mirror and actuates partial tilt/rotation of the mirror. Only one delay is necessary, although more than one delay line is possible. Alternatively, one can use a diffraction grating having a period which changes as a function of time to make the mirror fixed and not rotating. Simple, blazed, or other grating known to those of ordinary skill in the art, can be used. The grating sends different wavelengths to a lens and a galvanometric scanning mirror which alters the optical delay in the reference arm as a function of mirror angle.

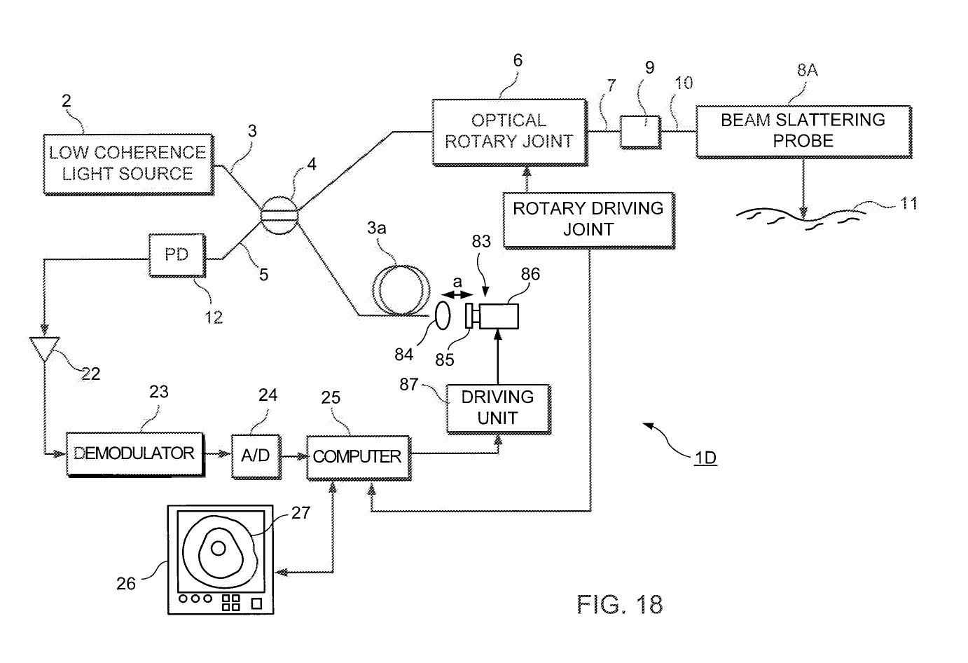

FIG. 18 is a schematic diagram of an alternative system in which the delay line is created by a mirror 84 is reciprocatingly mounted on a linear translator 85 which is controlled by a motor/driving unit 86 and 87. A description of basic components FIG. 18 is found in the specification corresponding to FIG. 11 of Ozawa et al. The mirror 84 oscillates at a certain rate. According to the present invention, the algorithms would have the mirror 84 scan back and forth and gradually shifts its translation over time to track the surface of the tissue. Each time the mirror 84 scans, it is called one scan or one axis of probing.

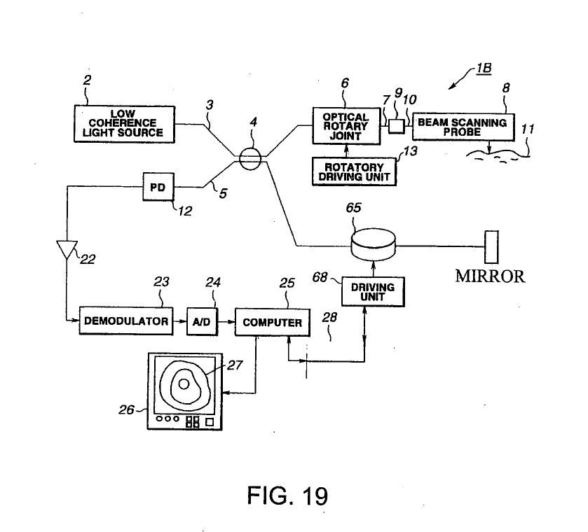

FIG. 19 (similar to FIG. 6 of Ozawa et al.) is a schematic diagram showing a further alternative system in which a drum 65 controlled by a computer 25. Small changes to the diameter of the drum, induced by piezoelectrics, stretch the thin fibers wound around the drum. The increased fiber length contributes a delay line.

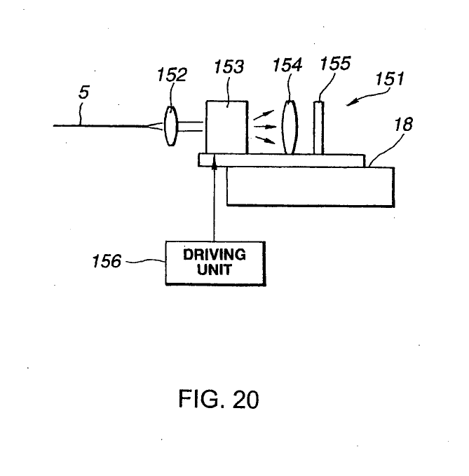

FIG. 20 illustrates an alternative using an acousto-optic modulator 153 is a computer controlled diffraction grating where the periodicity of the grating can be changed based on the frequency to the acousto-optic modulator.

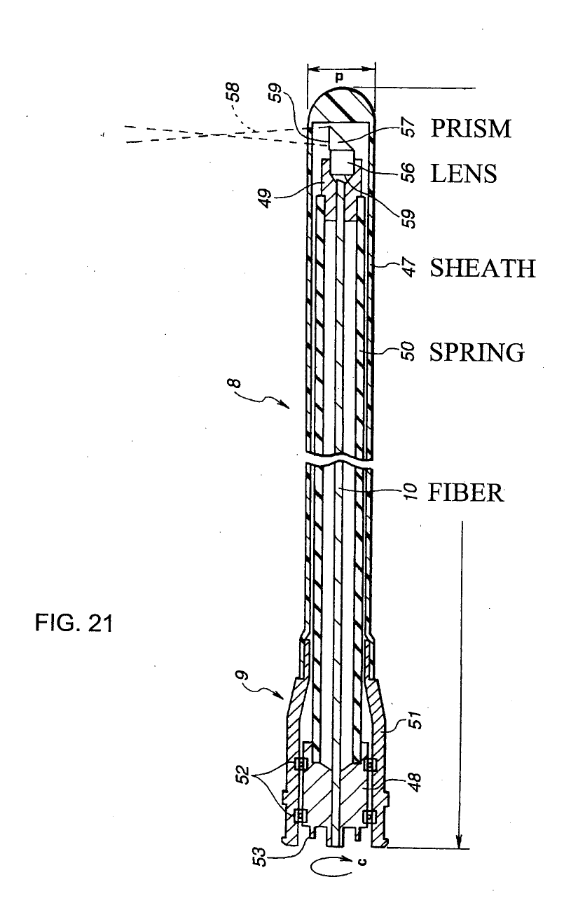

FIG. 21 shows a catheter according to the present invention, and is a modification of FIG. 4 of Ozawa et al.

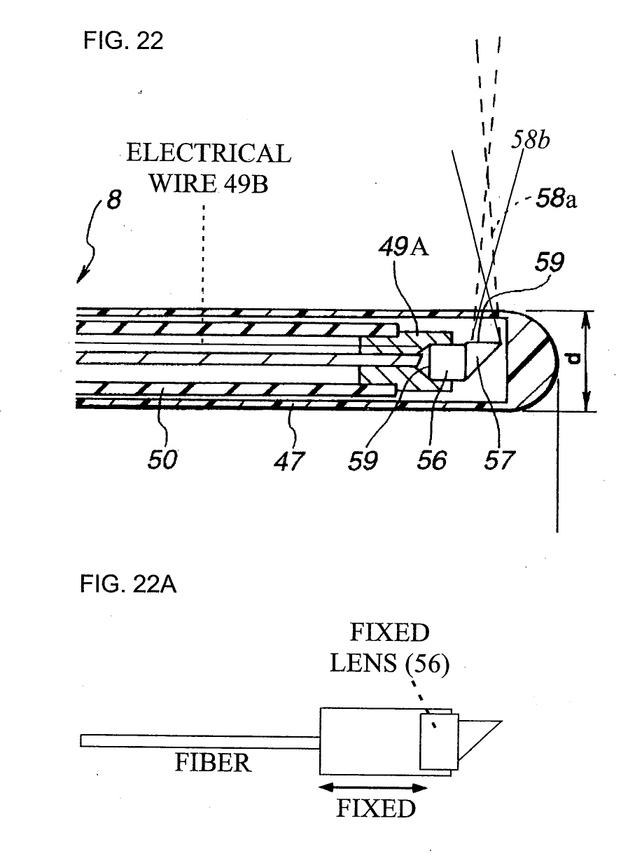

FIG. 22 shows a detail of a catheter according to one embodiment of the present invention. The design is based on FIG. 4 of Ozawa et al. FIG. 22A (a detail of FIG. 21) shows the distal end of the catheter having an optical fiber fixed into block 49, which fixes the fiber to the spring. Instead of a fixed block 49 the present invention uses a block which can have its length altered. In one embodiment, the block is a piezoelectric transducer (“piezo”) 49A connected by a wire 49B. The voltage changes the length of the piezo 49A and therefore changes the separation (the gap) between the lens 56 and the tip of the optical fiber. Movement of the lens with respect to the fiber tip is shown in the inset FIG. 22A. 58 is the output beam. 58a is the output beam at piezo voltage Va and 58b is the output beam at piezo voltage Vb.

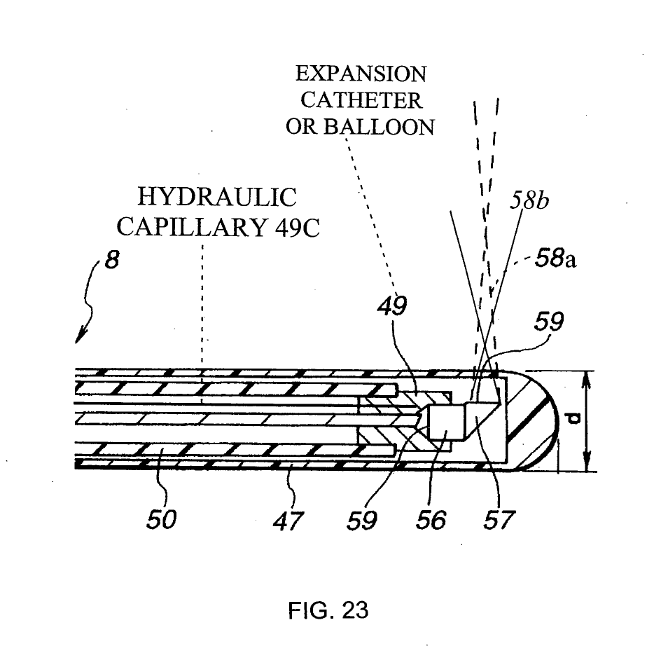

There are alternative ways to controllably change the distance between the lens and the fiber tip. One way is by using a balloon or an expansion chamber instead of the piezo 49. Instead of the wire 49B there is an air or hydraulic capillary 49C extending in the catheter 8. See FIG. 23, where 58a is the output beam at air or fluid pressure Pa and 58b is the output beam at pressure Pb.

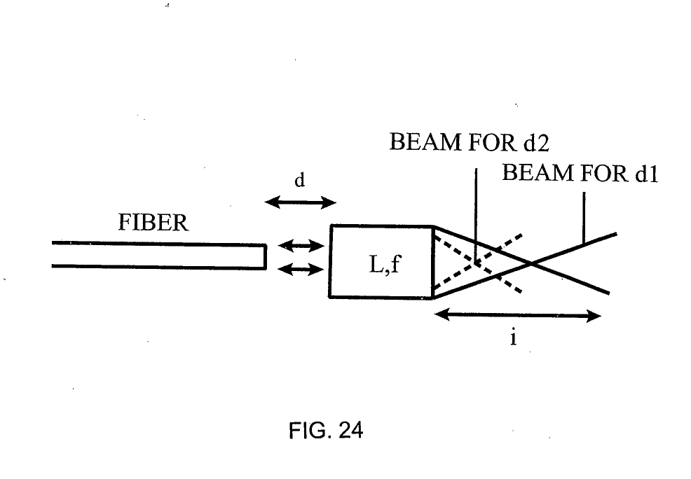

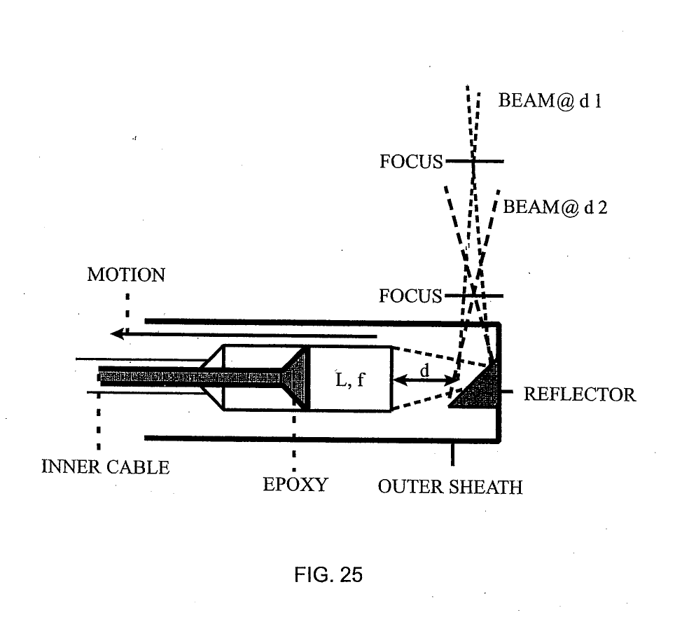

FIGS. 24 and 25 are two general ways to translate a focus. FIG. 24 shows a schematic view of a system which illustrates that as the distance between the fiber and the lens changes, the location of the focus changes. For object distance d₁ the focus is shown as a solid ray tracing line. For distance d₂ the focus is shown as the dashed ray tracing line. The relationship between distance and focal length is 1/d+1/i=1/f, where “i” is the image distance. Magnification M=i/d.

FIG. 25 shows a schematic view of a system where the fiber-lens separation is fixed and the separation between the lens and the reflector/prism is changed. In this embodiment, the light beam at distance d1 has a different focal point than the light beam at distance d2. The translation can be achieved by any of the mechanisms described above.

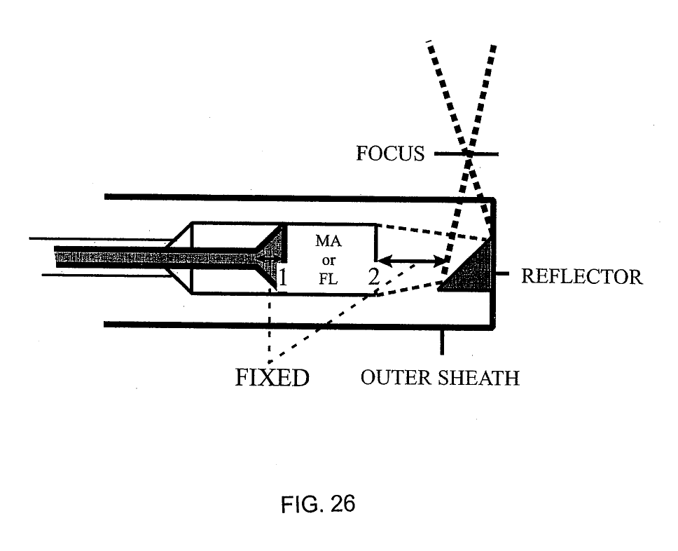

FIG. 26 shows a schematic view of a system where the gap between the fiber and a compound lens composed of multiple elements is fixed and, e.g., the gap between the lens and the reflector is fixed, but the relative separation of the gap between individual lens elements changes. An alternative embodiment utilizes a lens having a flexible cover and filled with an optically transparent fluid (e.g., saline, oil), gas or other substance. As the fluid composition, flexible cover shape or the like is changed, the focal length also changes.

It will be understood that the terms “a” and “an” as used herein are not intended to mean only “one,” but may also mean a number greater than “one.” While the invention has been described in connection with certain embodiments, it is not intended to limit the scope of the invention to the particular forms set forth, but, on the contrary, it is intended to cover such alternatives, modifications, and equivalents as may be included within the true spirit and scope of the invention as defined by the appended claims. All patent, applications and publications referred to herein are incorporated by reference in their entirety.

1-30. (canceled)

31. An apparatus for obtaining information associated with at least one structure, comprising:
at least one first arrangement configured to receive at least one first electromagnetic radiation from a first portion of the at least one structure which has a transverse dimension; and
at least one second arrangement configured to control a focal distance of at least one second electromagnetic radiation which is at least one of transmitted to or received from a second portion of the at least one structure as a function of the at least one first electromagnetic radiation,
wherein at least one of the first portion or the second portion has a transverse dimension of less than 10 μm.

32. The apparatus according to claim 31, wherein at least one of the at least one first arrangement or the at least one second arrangement is a confocal microscopy arrangement.

33. The apparatus according to claim 31, wherein at least one of the at least one first arrangement or the at least one second arrangement is a spectrally-encoded microscopy arrangement.

34. The apparatus according to claim 31, wherein at least one of the at least one first arrangement or the at least one second arrangement is provided in an expansion arrangement.

35. The apparatus according to claim 34, wherein the expansion arrangement is a balloon.

36. The apparatus according to claim 31, wherein the at least one second arrangement comprises a piezo-electric transducer.
